(19) United States
(12) Patent Application Publication
(10) Pub. No.: US 2026/0284661 A1
(43) Pub. Date: Sep. 24, 2026

(54) DEVICE AND PROCESS FOR RECOVERING ORGANELLE VESICLES FROM CELLS, USING A MICROFLUIDIC EXTRACTION UNIT

(71) Applicants: PARIS SCIENCES ET LETTRES, PARIS (FR); CENTRE NATIONAL DE LA RECHERCHE SCIENTIFIQUE, PARIS (FR); SORBONNE UNIVERSITE, PARIS (FR); UNIVERSITE PARIS CITE, PARIS (FR)

(72) Inventors: Abdou Rachid THIAM, PARIS (FR); Alexandre SANTINHO, PARIS (FR); Vincent FAUGERAS, Paris (FR)

(73) Assignees: PARIS SCIENCES ET LETTRES, PARIS (FR); CENTRE NATIONAL DE LA RECHERCHE SCIENTIFIQUE, PARIS (FR); SORBONNE UNIVERSITE, PARIS (FR); UNIVERSITE PARIS CITE, PARIS (FR)

(21) Appl. No.: 19/471,727

(22) PCT Filed: Apr. 9, 2024
(86) PCT No.: PCT/EP2024/059556

§ 371 Date: Oct. 2, 2025

(30) Foreign Application Priority Data
Apr. 11, 2023 (EP) 23305533.4

(51) Int. Cl. B01L 3/00; C12M 1/00; C12M 3/06

(52) U.S. Cl. CPC B01L 3/502746; B01L 3/502761; C12M 23/16; C12M 47/06; B01L 2200/0663; B01L 2300/0816; B01L 2300/0864; B01L 2400/0487; B01L 2400/086

(57) ABSTRACT

Device for recovering organelle vesicles from cells, including a microfluidic chip to extract organelle vesicles from cells, the microfluidic chip having one or more microchannels including one or more constriction portions. Process implementing such a device and extracellular organelle vesicles obtained by such a process.

### TECHNICAL FIELD

The present invention relates generally to devices and processes for recovering intracellular constituents from cells.

More specifically, the present invention refers to a microfluidic extraction unit.

### BACKGROUND

Recovering intracellular constituents from cells generally requires cell lysis, i.e. breaking the outer cell membrane to release said intracellular constituents with man-dependent protocols.

Cell lysis techniques known in the prior art include chemical, mechanical, electrical, thermal, and laser lysis processes.

Known techniques are costly in terms of human time, tedious, non-automatable, non-reproducible, generate very little biological material, and therefore prevent industrialization and data robustness.

### SUMMARY

In an industrialization perspective, the invention provides a device for recovering organelle vesicles from cells, comprising:
an organelle swelling unit configured to constitute swelled intracellular organelle vesicles, and
an extraction unit configured to extract swelled organelle vesicles from cells, said extraction unit comprising a microfluidic chip having one or more microchannels configured to receive a fluid flow containing said cells, said microchannels comprising one or more constriction portions configured to apply mechanical constraints on said cells while passing therethrough.

Such a microfluidic chip makes it possible to extract organelle vesicles from cells at high frequency, to control lysis parameter to minimize organelle damages to recover highly functional organelles vesicles and to have a reproducible process of extraction where each cell will be subjected to the same stress, being thus compatible with industrial requirements: volume production, organelle viability and reproducibility.

In one embodiment, at least one of said constriction portions is connected to an upstream portion and/or a downstream portion of a same microchannel.

Said upstream portion preferably has a section which decreases, preferably monotonically, from an upstream part of the microchannel to said constriction portion.

Said downstream portion preferably has a section which increases, preferably monotonically, from said constriction portion to a downstream part of the microchannel.

In one embodiment, said upstream portion is formed by:
at least one first wall member defining with a transverse plane an angle ranging from 91° to 179°, preferably from 99° to 170°, for instance 105° or 130°, and/or
at least one second wall member defining with said transverse plane an angle ranging from 90° to 170°, preferably from 90° to 160°, for instance 95° or 120°.

In one embodiment, said downstream portion is formed by:
at least one first wall member defining with a transverse plane an angle ranging from 91° to 180°, preferably from 120° to 179°, for instance 150° or 170°, and/or
at least one second wall member defining with said transverse plane an angle ranging from 90° to 170°, preferably from 90° to 160°, for instance 95° or 120.

When a constriction portion is connected to both an upstream portion and a downstream portion, it is preferred that said upstream portion is longer than said downstream portion, in relation to the direction of fluid flow.

In one embodiment, said constriction portion has:
a section ranging from 100 μm² to 2000 μm², preferably from 170 μm² to 650 μm², for instance 225 μm² or 330 μm², and/or
a width ranging from 0.5 μm to 15 μm, preferably from 2 μm to 15 μm, preferably from 4 μm to 15 μm, preferably from 7 μm to 13 μm, for instance 8, 9, 10, 11 or 12 μm, and/or
a height ranging from 0.5 μm to 15 μm, preferably from 2 μm to 100 μm, preferably from 5 μm to 100 μm, preferably from 10 μm to 30 μm, for instance 25, 30, 35 or 40 μm, and/or
a length ranging from 1 μm to 200 μm, preferably from 10 μm to 70 μm, preferably from 25 μm to 50 μm, for instance 20, 30, 40, 50 or 60 μm.

At least one of said constriction portions may have a polygonal cross-section, for instance a rectangular or trapezoidal cross-section.

In one embodiment, at least one of said microchannels comprises several of said constriction portions, for example two or more constriction portions.

In an embodiment, the constriction section is decreased from one to another constriction portion of a same microchannel, in relation to the fluid flow direction.

In one embodiment, several of said microchannels comprises one or more of said constriction portions, respectively.

In one embodiment, said organelle swelling unit comprises a means to put the cells in contact with a hypotonic aqueous medium.

In one embodiment, said organelle swelling unit comprises a means to put cells in contact with chemical compounds, for example to vesiculate one or more organelles for a duration ranging from 0.5 h to 48 h, preferably between 1 h to 36 h, more preferably between 2 h to 24 h.

In one embodiment, said organelle swelling unit comprises a means to put the cells in contact with vacuolin-1 at a concentration between 1 nmol/L to 100 μmol/L, preferably between 10 nmol/L to 5 μmol/L, more preferably between 100 nmol/L to 2 μmol/L for 1 hour to 48 hours, preferably between 2 hours to 24 hours or methylamine or rapamycin or bafilomycin to swell lysosomes and/or endo-lysosomes and/or endosomes and/or autophagosomes into large vesicles.

In one embodiment, said organelle swelling unit comprises a means to put the cells in contact with Actinomycin D, AZD 5582, AT 101, Camptothecin, Cisplatin, Doxorubicin, Etoposide, Mitomycin C MSC 2032964A, Nutlin 3, Paclitaxel, PRIMA-1MET, Staurosporine, Vinblastine, for example to induce cell apoptosis to induce the formation of large vesicles of several organelles.

In one embodiment, said organelle swelling unit is formed by a part of said microfluidic chip and/or includes an additional microfluidic chip.

In one embodiment, the device further comprises a collecting unit configured to collect and/or to sort said organelle vesicles extracted from cells with said extraction unit.

In one embodiment, said collecting unit is formed by a part of said microfluidic chip and/or includes an additional microfluidic chip.

In one embodiment, the device further comprises an observation unit configured to observe said organelle vesicles extracted from cells with said extraction unit.

In one embodiment, said observation unit includes a camera and/or a microscope, for example a confocal microscope.

Another object of the present invention is a process for recovering organelle vesicles from cells, comprising:
a step of swelling said organelle vesicles with an organelle swelling unit of a device as defined above, and
a step of extraction of swelled organelle vesicles from cells with an extraction unit of a device as defined above.

In one embodiment, the process comprises, after the extraction step, a step of collecting said organelle vesicles with a collecting unit as defined above.

In one embodiment, the process comprises an introduction of a fluid containing said cells within said microfluidic chip microchannels of said extraction unit with a flow rate ranging from 1 μL/min to 500 μL/min, preferably from 50 μL/min to 200 μL/min, for instance 75, 100, 125, 150 or 175 μL/min.

In one embodiment, the process further comprises, during and/or after the step of extraction, a step of observation of said organelle vesicles with an observation unit as defined above.

Another object of the present invention consists in extracellular organelle vesicles obtained by a process as defined above.

In one embodiment, said organelle vesicles have a size ranging from 3 μm to 15 μm, for example from 4 μm to 10 μm.

Of course, the above embodiments can be combined with each other.

The invention makes it possible to produce large quantities of organelle vesicles to make purified and concentrated samples that reach the market. The microfluidic extraction according to the invention is ideal to reach flow rates of typically a batch of a million of large organelle vesicles extracted per hour continuously. Moreover, the high frequency extraction unit of the invention can be directly connected to a sorting machine.

The present invention, and all its aspects, embodiments and advantages associated therewith will become more readily apparent in view of the detailed description of particular embodiments provided below, including the accompanying drawings.

### BRIEF DESCRIPTION OF THE DRAWINGS

In the following, non-limiting embodiments of the invention are described with reference to the accompanying schematic drawings in which:

FIG. 1 is a schematic representation of a device according to the invention, comprising a preparation unit, an extraction unit, a collecting unit, and an observation unit;

FIG. 2 is a schematic representation of a microfluidic chip according to the invention;

FIG. 3 is a schematic representation of a microchannel of the invention according to a first sectional plane;

FIG. 4 is a schematic representation of the microchannel of FIG. 3, according to a second sectional plane which is perpendicular to said first sectional plane;

FIG. 5 is a schematic representation of a microchannel according to an embodiment the invention;

FIG. 6 is a schematic representation of a microchannel according to another embodiment the invention;

FIG. 7 is a schematic representation of a microchannel according to a further embodiment the invention.

### DETAILED DESCRIPTION

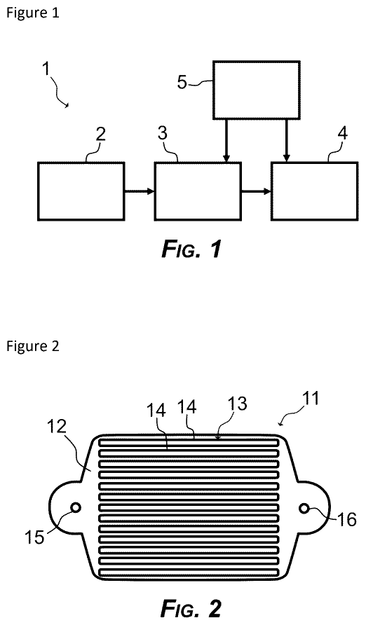

FIG. 1 illustrates schematically a device 1 according to a non-limiting embodiment of the invention. The device 1 is generally intended to recover organelle vesicles from cells.

In general, cells can be mammalian, plant and/or bacteria cells—e.g. COS-7 cells, Hela cells, HEK cells, CHO cells, fibroblast cells, red blood cells, T-cells, neuroblastoma cells, stem cells, iPSC (induced Pluripotent Stem Cells) derived into cell types such iPSC derived neuronal stem cells, iPSC derived mesenchymal stem cells, iPSC derived monocytes stem cells, iPSC derived cardiomyocyte stem cells, iPSC derived microglia stem cells, iPSC derived myotubes stem cells, etc.—obtained from the market, from laboratories and/or from hospitals, and/or recovered from organoids, biopsies, tissues, organs and/or organisms, from healthy or diseased human patient origin.

Organelles are defined here as differentiated biological material, such as lysosome, mitochondria, endoplasmic reticulum, Golgi apparatus, vacuole, chloroplast, autophagosome, autolysosome, endosome, endolysosome, peroxisome, multivesicular bodies.

The device 1 of FIG. 1 comprises a preparation unit 2 configured to prepare cells and intracellular organelle vesicles, an extraction unit 3 configured to extract organelle vesicles from cells, a collecting unit 4 configured to collect the extracted organelle vesicles, and an observation unit 5 configured to observe the extracted organelle vesicles.

### Preparation of Cells

In the present embodiment, the preparation unit 2 comprises a means (not shown) to put the cells in contact with a hypotonic aqueous medium, in order to constitute swelled and compartmented intracellular organelles, which are referred to as organelle vesicles.

The preparation unit 2 can thus comprise an organelle swelling unit, also called “swelling unit”, configured to produce such intracellular organelle vesicles, which may in particular be intracellular bilayer-bounded organelles, lipid droplets, ribosomes, or cytoskeleton. Preferably, organelle vesicles are not cell nuclei.

In this embodiment, the cells are put in contact with said hypotonic aqueous medium which can be any aqueous solution with an osmolarity ranging from 0.1 to 200 mOsm/L, preferably ranging from 0.1 to 100 mOsm/L, preferably from 1 to 50 mOsm/L, more preferably from 5 to 50 mOsm/L, the most preferably from 10 to 40 mOsm/L, during 0.5 to 30 minutes, for example during 0.5, 3, 5, 7, 10, 15, or 20 to 30 minutes.

As an example, the aqueous solution can derive from buffer solutions—e.g. diluted so-called “Dulbecco's Phosphate Buffered Saline” (DPBS)—, diluted cell culture medium—e.g. so-called “Dulbecco's Modified Eagle Medium” (DMEM)—, ionic solutions, salt solutions—e.g. CaCl2) or KCl solutions. Those types of solutions allow to obtain stable and functional organelle vesicles.

In an example protocol, Cos7, HeLa, HEK, CHO and fibroblast cells are maintained in DMEM supplemented with 10% heat inactivated Fetal Bovine Serum and 1% penicillin-streptomycin. Before the swelling step, cells are cultivated 48 h in DMEM media at 37° C. with 5% CO₂. Cells are transfected 24 h with the indicated plasmids to probe different organelles before extraction and recovery, and cultured left adhering in Matek dishes, with or without pre-treatment with adhesion agents.

According to this example protocol, cells are transfected with different plasmids fused with fluorescent protein constructs—e.g. red fluorescent protein (RFP) or blue fluorescent protein (BFP)—24 h before organelle vesicles' extraction. These plasmids serve to express proteins reporting for different organelles. Kdel-RFP and Mito-BFP plasmids can be used to identify the endoplasmic reticulum and the mitochondria.

The cell culture media can be diluted, before cells become confluent, with H₂O, pH 7.4, at 37° C. and 5% CO₂. Of course, the osmolarity gradient of the swelling solution can be controlled and adjusted depending on the cell type.

For indication purpose, in one particular embodiment in which cells are COS-7 cells, cytoplasm osmolarity is around 300 mOsm/L. Therefore, in such an embodiment, any aqueous solution having an osmolarity below about 100 mOsm/L and higher than 0.1 mOsm/L can be used to produce organelle vesicles.

The above-mentioned types of hypotonic aqueous medium enable to instantly apply to cells and its intracellular organelles an adequate non-destructive, fast and effective osmotic shock to generate spherical swollen cells and enlarged organelle vesicles.

Some molecules can be added to promote cytoskeleton disassembling and reducing the lysis tension of the cells, for example cytoskeleton disruptor such as nocodazole, navelbine, latrunculin A, latrunculin B and cytochalasin, and/or kinesin, myosin and/or dinein motor inhibitors such as blebistatin, benzytoluen sulphonamide, butanediome and monoxime.

Said hypotonic aqueous medium can also comprise one or more molecules to modulate the surface-to-volume ratio distribution of extracellular organelle vesicles while preventing their degradation and the value of surface tensions to lyse the plasma membrane, e.g. protease inhibitors, molecular motors inhibitors, organelle-cytoskeleton contact inhibitors, cytoskeleton disruptors, detergents.

Some molecules can also be added to accelerate cell swelling, e.g. ion channel modulator/blocker such as thaspsigargin, caffein and benzothiasepin, extra-cellular matrix distruptor such as trypsin, protein transport inhibitors, protein signaling inhibitors such as xelospongin, chemical detergents such as Triton-X-100, octyl-glucoside, DDM and/or carboxylic acids.

Said hypotonic aqueous medium can comprise one or more molecules selected from a group comprising nocodazole, latrunculins, trypsin, misakinolides, mycalolides, aplyronides, vinblastine, rotenone, swinholides, jasplakinolides, vincristine, demecolcine, cytochalasins, colchicine, vinca-alcaloids, dihydropyridine, phenylalkylamine, benzothiazepine, gabapentinoids, blebistatin, benzytoluen sulphonamide, butanediome monoxime, thaspsigargin, xelospongin, Triton X-100, Tween, SDS, Brij, Octyl Glucoside, octyl thioglucoside, CHAPS, CHAPSO, and magnesium.

The preparation unit 2 can thus be configured to generate said intracellular organelle vesicles with a decreased surface-to-volume ratio—surface divided by the volume of a geometric shape, spherical in most cases—compared to their original form, for example a surface-to-volume ratio ranging from 0.15 μm⁻¹ to 3 μm⁻¹, preferably from 0.15 μm⁻¹ to 2 μm⁻¹, more preferably from 0.15 μm⁻¹ to 1.5 μm⁻¹, most preferably between 0.15 μm⁻¹ to 1.2 μm⁻¹, and/or having a size greater than 4 μm within the cells. Such organelle vesicles can thus be referred to as “giant” organelle vesicles.

In an embodiment, the preparation unit 2 comprises an additional means (not shown) to treat the cells before putting them in contact with said hypotonic aqueous medium, in particular so that extracellular organelle vesicles can bear specific biochemical properties. For example, this treatment can be done to prevent protein degradation and/or to modulate biochemical reactions on organelles. In other words, the metabolic conditions and the architecture of cells and its organelles can be modified by chemicals and/or by modifying the expression level of proteins impacting the architecture of cells and/or their organelles and/or the metabolic conditions and/or the surface-to-volume ratio and/or the relative location and/or arrangement of the organelles in relation to one another.

For example, prior to the above-described osmotic swelling step, the surface-to-volume ratio of the future produced extracellular organelle vesicles can be modulated:
by altering the expression levels of proteins impacting organelle shape and contact sites with other organelles, plasma membrane and cytoskeleton, and/or by treating cells with molecules that alter both the cytoskeleton and molecular motors, organelle contacts sites—with other organelles, plasma membrane and cytoskeleton—, molecules-transporting-proteins activity localized on plasma membranes and organelles, signaling protein activity, and/or
by altering cellular metabolic pathways impacting organelles number and/or shape, and/or
with any treatment mediate change in organelle surface-to-volume ratio and/or inter-organelle contact.

For example, overexpressing CLIMP-63 before osmotic swelling may result in larger extracellular organelle vesicles emanating from the endoplasmic reticulum with sizes larger than 30 μm. In the same way, overexpressing Mfn2 before osmotic swelling may result in larger extracellular organelle vesicles emanating from the mitochondria with surface-to-volume ratio smaller than 0.75 μm⁻¹. Pre-treating cells with nocodazole, or Latrunculin A, between 1 and 90 min before swelling allows the formation of bigger organelles from the endoplasmic reticulum. Adding rapamycin between 12 to 24 hours before swelling formation allows to form bigger extracellular organelle vesicles from endosome, lysosome, autolysosome and multivesicular body. Adding bafylomicin before swelling also allows to obtain more extracellular organelle vesicles coming from autophagosomes.

In a further embodiment, the preparation unit 2 comprises an additional means (not shown) to treat the cells after osmotic swelling, by generating a back-and-forth motion of the hypotonic aqueous medium to displace cells, for example at a speed ranging from about 0.01 m/s to 10 m/s during about 0.01 second to 10 minutes, to disrupt both cytoskeleton and extracellular matrix of cells. This optional treatment allows reducing the required mechanical constraints to be applied to the cells in said extraction unit 3 (see below) to lyse cells. Indeed, the cytoskeleton gives the plasma membrane of the cell an additional resistance. Therefore, subjecting the cells to such a motion, without lysing them, allows to lower the further lysis tension without damaging the intracellular organelle vesicles still inside.

Extracting Organelle Vesicles from Cells

According to the invention, said extraction unit 3 comprises a microfluidic chip 11, as shown in the non-limiting example of FIG. 2.

As known per se, the microfluidic chip 11 comprises a body 12 made of a material such as Polydimethylsiloxane (PDMS), and an array 13 of microchannels 14, in this example thirteen microchannels 14, formed by the body 12 and fluidly connected to at least one inlet 15 and at least one outlet 16.

An example of manufacturing of the chip 11 is described further down below. Of course, the body 12, the chip 11 can be manufactured using any technique and/or material.

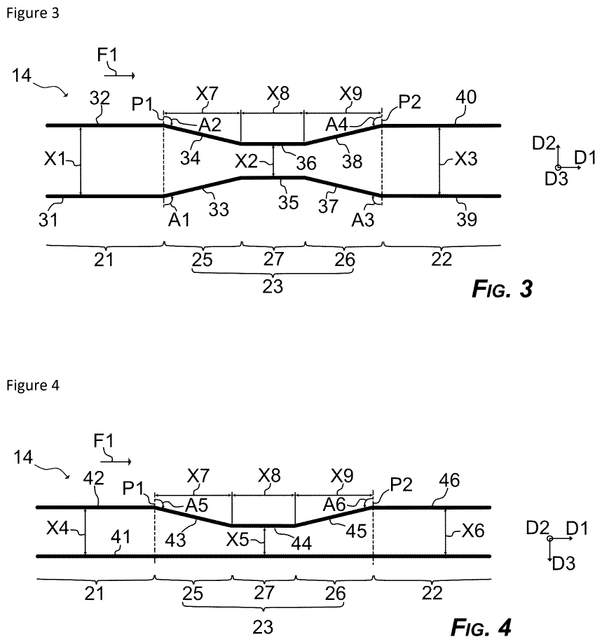

FIGS. 3 and 4 illustrate schematically a longitudinal portion of a microchannel 14 according to the invention, and provide a frame of reference defining a longitudinal direction D1 and transversal directions D2 and D3, the D1, D2 and D3 directions being orthogonal to one another. FIG. 3 is a view of said microchannel 14 portion according to a D1-D2 sectional plane. FIG. 4 is a view of the same microchannel 14 portion according to a D1-D3 sectional plane.

In the present description, the words “upstream” and “downstream” are referring to a flow direction F1 along D1 of a fluid flowing into the microchannel 14 during the extraction step.

The longitudinal portion of the microchannel 14 shown in FIGS. 3 and 4 generally comprises an upstream part 21, a downstream part 22 and, between said upstream part 21 and downstream part 22, an intermediate part 23.

Said intermediate part 23 of the microchannel 14 itself comprises different parts along direction D1, namely an upstream portion 25, a downstream portion 26 and, between said upstream portion 25 and downstream portion 26, an intermediate portion 27.

In the present embodiment, the microchannel 14 of FIGS. 3 and 4 is defined by wall members 31-46.

With reference to FIG. 3, wall members 31, 33, 35, 37 and 39 define a first lateral surface of the microchannel 14, and wall members 32, 34, 36, 38 and 40 define a second lateral surface of the microchannel 14. First and second lateral surfaces are spaced from each other in the direction D2.

With reference to FIG. 4, wall member 41 define a third lateral surface of the microchannel 14, and wall members 42, 43, 44, 45 and 46 define a fourth lateral surface of the microchannel 14. Third and fourth lateral surfaces are spaced from each other in the direction D3.

In this embodiment, each of the wall members 31-40 extends parallel to the direction D2, and each of the wall members 41-46 extends parallel to the direction D2.

Therefore, the portion of the microchannel 14 shown in FIGS. 3 and 4 has in this case a rectangular or square-shaped transverse section, also called cross-section, at any coordinate along the direction D1.

As illustrated in FIGS. 3 and 4, the section of said intermediate portion 27 is smaller than the section of said upstream part 21 and downstream part 22, given the respective orientation of said wall members.

In this embodiment, wall members 31, 32, 35, 36, 39, 40, 41, 42, 44 and 46 are parallel to the direction D1, while wall members 33, 34, 37, 38, 43 and 45 are oblique to the direction D1.

More specifically, in reference to FIG. 3, wall member 33 defines an angle A1 with a fictitious transverse plane P1 which is perpendicular to the direction D1 and which delimits said upstream part 21 from said intermediate part 23 of the microchannel 14. Wall member 34 also defines an angle A2 with said transverse plane P1.

Similarly, wall member 37 defines an angle A3 with a fictitious transverse plane P2 which is perpendicular to the direction D1 and which delimits said downstream part 22 from said intermediate part 23 of the microchannel 14. Wall member 38 also defines an angle A4 with said transverse plane P2.

In reference to FIG. 4, wall member 43 defines an angle A5 with said transverse plane P1, and wall member 45 defines an angle A6 with said transverse plane P2.

In this embodiment, the wall members 33, 34, 41 and 43 are configured so that the section of said upstream portion 25 monotonically decreases from said upstream part 21 of the microchannel 14 to said intermediate portion 27 of the intermediate part 23 of the microchannel 14. Similarly, the wall members 37, 38, 41 and 45 are configured so that the section of said downstream portion 26 monotonically increases from said intermediate portion 27 of the intermediate part 23 of the microchannel 14 to said downstream part 22 of the microchannel 14.

The term “monotically” is to be understood in the mathematical sense, a monotonic function being defined as a function whose first derivative does not change sign.

In this particular embodiment, said upstream portion 25 and said downstream portion 26 are symmetrical with respect to a fictitious transverse plane (not shown) extending equidistant to the planes P1 and P2.

In light of the above and given that the section of said intermediate portion 27 is smaller than the section of said upstream part 21 and downstream part 22, the intermediate portion 27 forms a portion of the microchannel 14 called “constriction portion”.

With reference to FIGS. 3 and 4, the upstream part 21, the constriction portion 27 and the downstream part 22 of the microchannel 14 has a first size according to the direction D2 respectively indicated by references X1, X2 and X3, and a second size according to the direction D3 respectively indicated by references X4, X5 and X6. Said first and second sizes are also called width and height, respectively.

In addition, the upstream portion 25, the constriction portion 27 and the downstream portion 26 of the microchannel 14 has a size according to the direction D1, also called length, respectively indicated by references X7, X8 and X9.

The non-limitative above-described geometric parameters can have the following values:
X1: ranging from 30 μm to 200 μm, preferably from 50 μm to 150 μm, for instance 100 μm;
X2: ranging from 4 μm to 15 μm, preferably from 7 μm to 13 μm, for instance 8, 9, 10, 11 or 12 μm;
X3: ranging from 30 μm to 200 μm, preferably from 50 μm to 150 μm, for instance 100 μm;
X4: ranging from 10 μm to 100 μm, preferably from 20 μm to 50 μm, for instance 30 μm;
X5: ranging from 5 μm to 100 μm, preferably from 10 μm to 30 μm, for instance 25, 30, 35 or 40 μm;
X6: ranging from 10 μm to 100 μm, preferably from 20 μm to 50 μm, for instance 30 μm;
X7: ranging from 5 μm to 500 μm, preferably from 50 μm to 300 μm, for instance 200 μm;
X8: ranging from 1 μm to 200 μm, preferably from 10 μm to 70 μm, preferably from 25 μm to 50 μm, for instance 20, 30 μm or 40 μm;
X9: ranging from 1 μm to 500 μm, preferably from 5 μm to 200 μm, for instance 10, 30, 50, 70, 90, 110, 130, 150, 170 or 190 μm;
A1: ranging from 91° to 179°, preferably from 99° to 170°, for instance 105° or 130°;
A2: ranging from 91° to 179°, preferably from 99° to 170°, for instance 105° or 130°;
A3: ranging from 91° to 180°, preferably from 120° to 179°, for instance 150° or 170°;
A4: ranging from 91° to 180°, preferably from 120° to 179°, for instance 150° or 170°;
A5: ranging from 90° to 170°, preferably from 90° to 160°, for instance 95° or 120°;
A6: ranging from 90° to 170°, preferably from 90° to 160°, for instance 95° or 120°.

Obviously, said upstream part 21, downstream part 22 and intermediate part 23, as well as said upstream portion 25, downstream portion 26 and constriction portion 27, are fluidically connected to each other, so that a fluid entering in the upstream part 21 can flow through the microchannel 14 in said flow direction F1 across these different parts and portions of the microchannel 14.

In the present embodiment, the wall members 31-46 are formed in one piece from a same material, for example forming the body of a microfluidic chip such as the chip 11 of FIG. 2.

Referring again to FIG. 2, in one embodiment, each of said microchannels 14 of the chip 11 comprises one microchannel portion similar to the one described above in reference to FIGS. 3 and 4. In another embodiment, each of said microchannels 14 of the chip 11 comprises several microchannel portions similar to the one described above in reference to FIGS. 3 and 4, thus forming series of constriction portions 27.

Of course, the chip 11 of FIG. 2, or any other type of microfluidic chip, may comprise one or more microchannels having one or more portions similar to the one described above in reference to FIGS. 3 and 4 and/or having other geometrical features. In particular, the size and/or the shape of the constriction portion 27 and/or of any other part or portion of the microchannel 14 may be different from those described above. Specifically, the above-described geometric parameters of the microchannel 14 can be combined with different respective values.

Furthermore, the constriction portion 27 and/or of any other part or portion of the microchannel 14 may have a cross-section which is not rectangular or square-shaped, e.g. a trapezoidal cross-section or other polygonal cross-section.

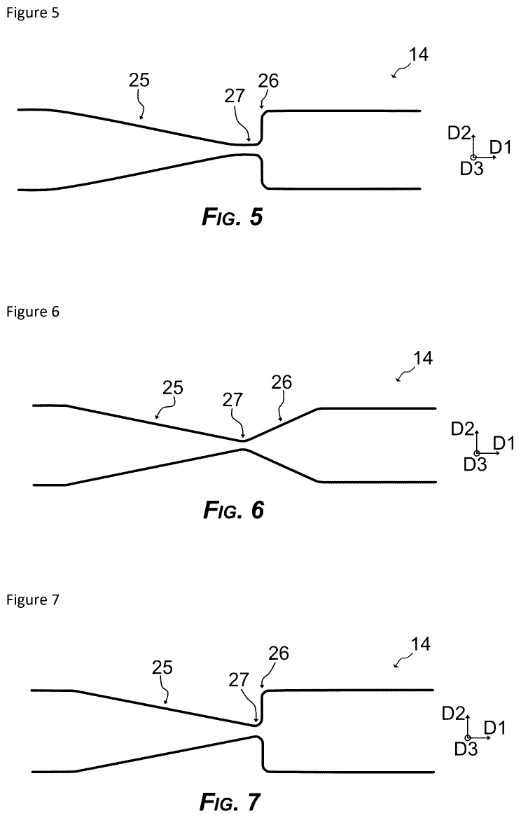

In some embodiments, as those illustrated in FIGS. 5-7, said upstream portion 25 is longer than said downstream portion 26. As a non-limitative indication, the ratio between upstream portion 25 and downstream portion 26 is about 2.4 in the embodiment of FIG. 6 and higher than 50 in the embodiments of FIGS. 5 and 7.

The above description applies by analogy to the embodiments of FIGS. 5-7.

In the embodiment of FIG. 5, the microchannel 14 has the following geometric parameters: X1=X3=100 μm; X2=13 μm; X4=X5=X6=25 μm; X8=38 μm; A1=A2=110°; A3=A4=1°; A5=A6=90°.

In the embodiment of FIG. 6, the microchannel 14 has the following geometric parameters: X1=X3=100 μm; X2=7 μm; X4=X5=X6=25 μm; X8=9 μm; A1=A2=111°; A3=A4=64°; A5=A6=90°.

In the embodiment of FIG. 7, the microchannel 14 has the following geometric parameters: X1=X3=100 μm; X2=11 μm; X4=X5=X6=25 μm; X8=6 μm; A1=A2=100°; A3=A4=1°; A5=A6=90°.

With reference to the embodiment of FIG. 1, the microfluidic chip 11 of said extraction unit 3 is in this example connected to said preparation unit 2 to receive within the microchannels 14 a fluid flow containing said cells and intracellular organelle vesicles.

Said fluid can be introduced within the microchannels 14 with a flow rate of about 100 μL/min and at a concentration between 10⁵ cells to 10⁷ cells per mL, for example.

The constriction portions 27 of the microchannels 14 apply mechanical constraints on said cells while passing therethrough, allowing in this example to lyse and remove cell plasma membranes, and to the release in the hypotonic medium of stable and functional extracellular organelle vesicles, without lysing the latter.

Therefore, the device 1 and in particular the extraction microfluidic unit 3 allows to generate extracellular organelle vesicles, typically with a mean size generally ranging from about 3 to 15 μm.

### Collecting Extracted Organelle Vesicles

In this example, the collecting unit 4 of the device 1 is configured to sort the organelle vesicles according to their type and to extract separately membrane fragments coming out from said extraction unit 3.

In an embodiment, the collecting unit 4 comprises an additional microfluidic chip (not shown).

In another embodiment, one and the same microfluidic chip (not shown) forms both the microchannels for extracting organelle vesicles from cells, and further microchannels for sorting and collecting the extracted organelle vesicles. In other words, one and the same microfluidic chip can form both the extracting unit 3 and collecting unit 4, or parts of these units.

In embodiments where the collecting unit 4 comprises microchannels, those microchannels can be spiral microchannels known in the art.

In another embodiment, the collecting unit 4 is configured to purify organelle vesicles using a fluorescence-activated cell sorting (FACS) technique, which is also known in the art.

Different types of sorting and/or collecting techniques can be combined, including but not limited to microfluidic and FACS techniques.

### Observation of Organelle Vesicles

The observation unit 5 of the device 1 is configured to allow observation of the extracted organelle vesicles with any appropriate techniques.

In an embodiment, the observation unit 5 comprises an ultra-fast camera, for example configured to realize up to 10000 frames per second, which typically allows visualization in live of organelle vesicle extraction. For example, extraction recordings can be made at a resolution of 2000 to 5000 frames per second, to generate videos with a slow-motion factor of 80 to 210.

In an embodiment, the observation unit 5 comprises a confocal microscope, e.g. a microscope known as “LSM800 Zeiss”, which typically allows visualization of extracellular organelle vesicles coming out from said extraction unit 3.

The observation unit 5 can comprise a combination of different controlling devices, including but not limited to ultra-fast camera and/or confocal microscope.

### Microfluidic Chip Manufacturing

A microfluidic chip according to the invention can be fabricated as follows.

In a first step, a photomask is produced to manufacture wafers of the chip with a photolithography technique, using a SU-8 2025 or SU-8 2050 negative resin. Patterns with a thickness of 25 μm or 50 μm, respectively, can thus be obtained. The following table provide protocols for wafers having a thick of 25 μm and 50 μm:

| | 25 μm thick wafer | | 50 μm thick wafer | |
|---|---|---|---|---|
| Spin-coating | 3000 | rpm | 300 | rpm |
| Soft bake | 65° C. for a few seconds; | | 65° C. during 30 s; | |
| | 95° C. during 5 min | | 95° C. during 6 min | |
| Ultraviolet exposure | ~7 | s | ~7 | s |
| (23.3 mW/cm2) | | | | |
| Post exposure bake | 1 min at 65° C.; | | 1 min at 65° C.; | |
| | 5 min at 95° C. | | 6 min at 95° C. | |
| Propylene glycol methyl | 4 | min | 5 | min |

| | | |
|---|---|---|
| ether acetate (PGMEA) | | |
| development | | |
| Cleaning/drying | Isopropyl/air gun | Isopropyl/air gun |
| Hard bake | 15 min at 200° C. | 15 min at 200° C. |

A wafer thus obtained is subjected to a gas-phase methyltrichlorosilane treatment to stiffen the resin affixed to the wafer and facilitate the removal of the polymerized PDMS from the wafer. Thus, 15 μl of methyltrichlorosilane is deposited in a petri dish with the wafer. The petri dish is sealed tightly before the product evaporates too much using parafilm. The treatment takes about 15 min to allow the deposition of hydrophobic residues.

In this example, the PDMS chip is fabricated with elastomeric silicone marketed under the name “Sylgard 184” and its 184-polymerization initiator with an elastomer-crosslinker ratio of 10:1. The mixture is vigorously homogenized, degassed in a vacuum chamber and carefully deposited on the wafer surrounded by an aluminum mold. Once the PDMS is poured to the height, the whole assembly is put in an incubator at 70° C. during 2 h. Once the PDMS is cured, the aluminum mold is cut and the PDMS is removed from the wafer. The PDMS is then cut to the size of the desired chip and perforated at the level of the inlets and outlets provided. Once the PDMS parts are cut, drilled and cleaned, they are exposed to glass slides, an exposure to air plasma—plasma chamber air pressure between 0.6 and 0.8 mbar—for 1 min to allow an irreversible adhesion of the PDMS and the glass slide.

Before being used to extract organelle vesicles, the chip is preferably submitted to an air plasma exposure, to increase hydrophobicity.

1-15. (canceled)

16. A device for recovering organelle vesicles from cells, comprising:
an organelle swelling unit configured to constitute swelled intracellular organelle vesicles, and
an extraction unit configured to extract swelled organelle vesicles from cells, said extraction unit comprising a microfluidic chip having one or more microchannels configured to receive a fluid flow containing said cells, said microchannels comprising one or more constriction portions configured to apply mechanical constraints on said cells while passing therethrough.

17. The device according to claim 16, wherein at least one of said constriction portions is connected to an upstream portion and a downstream portion of a same microchannel, said upstream portion having a section which decreases, preferably monotonically, from an upstream part of the microchannel to said constriction portion, said downstream portion having a section which increases, preferably monotonically, from said constriction portion to a downstream part of the microchannel.

18. The device according to claim 17, wherein:
said upstream portion is formed by:
at least one first wall member defining with a transverse plane (P1) an angle (A1, A2) ranging from 91° to 179°, preferably from 99° to 170°, for instance 105° or 130°, and/or
at least one second wall member defining with said transverse plane (P1) an angle (A5) ranging from 90° to 170°, preferably from 90° to 160°, for instance 95° or 120°, and/or
said downstream portion is formed by:
at least one first wall member defining with a transverse plane (P2) an angle (A3, A4) ranging from 91° to 180°, preferably from 120° to 179°, for instance 150° or 170°, and/or
at least one second wall member defining with said transverse plane (P2) an angle (A6) ranging from 90° to 170°, preferably from 90° to 160°, for instance 95° or 120°.

19. The device according to claim 17, wherein said upstream portion is longer than said downstream portion, in relation to the direction (F1) of fluid flow.

20. The device according to claim 16, wherein said constriction portion has:
a section ranging from 100 μm² to 2000 μm², preferably from 170 μm² to 650 μm², for instance 225 μm² or 330 μm², and/or
a width (X2) ranging from 0.5 μm to 15 μm, preferably from 2 μm to 15 μm, preferably from 4 μm to 15 μm, preferably from 7 μm to 13 μm, for instance 8, 9, 10, 11 or 12 μm, and/or
a height (X5) ranging from 0.5 μm to 15 μm, preferably from 2 μm to 100 μm, preferably from 5 μm to 100 μm, preferably from 10 μm to 30 μm, for instance 25, 30, 35 or 40 μm, and/or
a length (X8) ranging from 1 μm to 200 μm, preferably from 10 μm to 70 μm, preferably from 25 μm to 50 μm, for instance 20, 30, 40, 50 or 60 μm.

21. The device according to claim 16, wherein at least one of said constriction portions has a polygonal cross-section, for instance a rectangular or trapezoidal cross-section.

22. The device according to claim 16, wherein at least one of said microchannels comprises several of said constriction portions.

23. The device according to claim 16, wherein several of said microchannels comprises one or more of said constriction portions, respectively.

24. The device according to claim 16, wherein said organelle swelling unit comprises a means to put the cells in contact with a hypotonic aqueous medium.

25. The device according to claim 16, further comprising a collecting unit configured to collect and/or to sort said organelle vesicles extracted from cells with said extraction unit, said collecting unit may be formed by a part of said microfluidic chip and/or may include an additional microfluidic chip.

26. The device according to claim 16, further comprising an observation unit configured to observe said organelle vesicles extracted from cells with said extraction unit, said observation unit may include a camera and/or a microscope.

27. A process for recovering organelle vesicles from cells, comprising:
a step of swelling said organelle vesicles with an organelle swelling unit of a device according to claim 1, and
a step of extraction of swelled organelle vesicles from cells with an extraction unit of the device, and
after the extraction step, a step of collecting said organelle vesicles with a collecting unit of the device.

28. The process according to claim 27, comprising an introduction of a fluid containing said cells within said microfluidic chip microchannels of said extraction unit with a flow rate ranging from 1 μL/min to 500 μL/min, preferably from 50 μL/min to 200 μL/min, for instance, 75, 100, 125, 150 or 175 μL/min.

29. The process according to claim 27, further comprising, during and/or after the step of extraction, a step of observation of said organelle vesicles with an observation unit of the device.

30. An extracellular organelle vesicles obtained by a process according to claim 27, said organelle vesicles having a size ranging from 3 μm to 15 μm, for example from 4 μm to 10 μm.
